(19) United States
(12) Patent Application Publication
(10) Pub. No.: US 2023/0073946 A1
(43) Pub. Date: Mar. 9, 2023

(54) AFFINITY PEPTIDE CONJUGATED WITH ANTIOXIDANT FOR PROTECTION OF PROTEINS FROM OXIDATION

(71) Applicant: ARIZONA BOARD OF REGENTS ON BEHALF OF THE UNIVERSITY OF ARIZONA, Tucson, AZ (US)

(72) Inventors: Ruslan Rafikov, Tucson, AZ (US); Olga Rafikova, Tucson, AZ (US)

(21) Appl. No.: 17/794,229

(22) PCT Filed: Jan. 20, 2021
(86) PCT No.: PCT/US21/14169

§ 371 Date: Jul. 20, 2022

Related U.S. Application Data
(60) Provisional application No. 62/963,260, filed on Jan. 20, 2020.

(51) Int. Cl. A61K 47/64; A61P 11/00; A61P 9/12; C12N 9/12

(52) U.S. Cl. CPC A61K 47/64; A61P 11/00; A61P 9/12; C12N 9/1205; A61K 38/00

(57) ABSTRACT

The present invention is a targeted peptide conjugated with an antioxidant, NO, to inhibit Akt nitration. The nitroxide peptide (NP), comprises two parts, one being the peptide part with the affinity to Akt near Tyr 350 residue (Ser-Arg-IIe-Arg-Ser; SRIRS)—and the other, a conjugated antioxidant—nitroxide (3-Carboxy-2,2,5,5-tetramethyl-3-pyrroline-1-yloxy) covalently attached to the free N-terminal amine. It can be utilized for treating pulmonary arterial hypertension (PAH) with potential application in other abnormal proliferative disorders such as cancer. The present invention features targeted selectivity for Akt nitration and does not affect Akt phosphorylation nor other physiological processes controlled by Akt signaling pathways. The present invention may also apply to other proliferative disorders including cancer or other disorders that cause or are caused by Akt nitration or protein oxidation.

### CROSS-REFERENCES TO RELATED APPLICATIONS

This application claims the benefit of U.S. Provisional Application No. 62/963,260 filed Jan. 20, 2020, the specification(s) of which is/are incorporated herein in their entirety by reference.

### STATEMENT REGARDING FEDERALLY SPONSORED RESEARCH OR DEVELOPMENT

This invention was made with government support under Grant Nos. R01 HL132918 and R01 HL133085 awarded by National Institutes of Health. The government has certain rights in the invention.

### REFERENCE TO A SEQUENCE LISTING

Applicant asserts that the information recorded in the form of an Annex C/ST.25 text file submitted under Rule 13ter.1(a), entitled, UNIA_19_21_PCT_Sequence_Listing_ST25, is identical to that forming part of the international application as filed. The content of the sequence listing is incorporated herein by reference in its entirety.

### FIELD OF THE INVENTION

The present invention is a targeted peptide conjugated with an antioxidant, NO, to inhibit Akt1 nitration. The nitroxide peptide (NP), comprises two parts, one being the peptide part with the affinity to Akt near Tyr 350 residue (Ser-Arg-IIe-Arg-Ser; SRIRS)—and the other, a conjugated antioxidant—nitroxide (3-Carboxy-2,2,5,5-tetramethyl-3-pyrroline-1-yloxy) covalently attached to the free N-terminal amine. It can be utilized for treating pulmonary arterial hypertension (PAH) with potential application in other abnormal proliferative disorders such as cancer. The present invention features targeted selectivity for Atk1 nitration and does not affect Atk1 phosphorylation nor other physiological processes controlled by Atk1 signaling pathways. The present invention may also apply to other proliferative disorders including cancer or other disorders that cause or are caused by Akt nitration or protein oxidation. Non-limiting examples of the proliferative disorders include atherosclerosis, rheumatoid arthritis, psoriasis, idiopathic pulmonary fibrosis, asthma, restenosis, and diabetic retinopathy.

### BACKGROUND OF THE INVENTION

Pulmonary arterial hypertension (PAH) is a disorder affecting 1.1 to 17.6 million adults per year with a prevalence of 6.6 to 26 million. This progressive disorder is characterized by angioproliferative vasculopathy in the pulmonary arterioles leading to endothelial and smooth muscle proliferation, dysfunction, inflammation and thrombosis. Currently, the disease remains incurable despite existing therapies, which are largely ineffective. Patients are therefore limited to invasive heart-lung transplantation which is only available in limited surgical centers worldwide and has only a 50% survival rate at 5 years.

PAH pathology is associated with the thickening/remodeling of the pulmonary artery wall. The Atk1 (Protein Kinase B) the serine/threonine kinase pathway plays an important role in normal and pathological vascular development and functioning as well as in other cellular functions such as glucose metabolism, cell proliferation, apoptosis, cell migration and transcription. Increased activation of the Atk1 signaling pathway is believed to play a role in PAH pathophysiology through inhibition of apoptotic signaling and promotion of cell survival. In particular, increased nitric oxide (NO) production, augmented high levels of peroxynitrite formation, nitrosative stress and enhanced protein nitration have been reported in PAH.

Antioxidant therapy has been studied for several decades, but it's cellular effects are global, and it does not show any clinical significance. Recently, it was reported that in sugen/hypoxic PAH model, the chemical antioxidant, TEMPOL, failed to attenuate right ventricular hypertrophy and pulmonary arterial remodeling. A targeted approach in antioxidant therapy is a promising technique and could maximize the potential of an antioxidant. Nitroxides are the family of free radical compounds, which are effective scavengers of reactive oxygen species (ROS). Nitroxides function as a superoxide dismutase (SOD) mimetic, performing the interconversion of an oxammonium cation or the hydroxylamine. These properties make nitroxide suitable for the development of defensive peptides for preventing Akt nitration.

The affinity peptide of the present invention that is exclusively targeted to protect the Akt nitration site, is conjugated with nitroxide moiety forming nitroxide-peptide (NP). Specifically, this would block the tyrosine (Tyr)-350 nitration of Akt, and the conjugated nitroxide antioxidant part would effectively alleviate free radical attacks. Without wishing to limit the present invention to any particular theory or mechanism, it is believed that preventing Akt nitration with antioxidant NP peptides is an effective remedy in controlling metabolic reprogramming, anaplerosis, and vascular remodeling events in PAH.

The present invention is a targeted peptide conjugated with an antioxidant, nitroxide, that acts as an inhibitor at the Atk1 nitration site associated with PAH pathological conditions. The present invention features a targeted treatment for PAH and other proliferative disorders. This targeted selectivity for Atk1 nitration will not affect Atk1 phosphorylation, and thus will not affect other physiological processes controlled by Atk1 signaling pathways.

### BRIEF SUMMARY OF THE INVENTION

It is an objective of the present invention to provide compositions and methods that allow for protection from oxidation and treatment of PAH, as specified in the independent claims. Embodiments of the invention are given in the dependent claims. Embodiments of the present invention can be freely combined with each other if they are not mutually exclusive.

The present invention features a conjugated peptide, a composition comprising the conjugated peptide, and methods of using the conjugated peptide for treatment of PAH. The conjugated peptide has affinity for Akt and inhibits nitration of Akt. The conjugated peptide comprises a peptide operably connected (e.g., conjugated, bound) to an antioxidant moiety allowing protection of proteins from oxidation.

One of the unique and inventive technical features of the present invention is a targeted peptide conjugated to an antioxidant that can inhibit or prevent protein oxidation of a targeted protein. In particular, the present invention features a conjugated peptide targeted to Akt to prevent Akt nitration. Without wishing to limit the invention to any theory or mechanism, it is believed that the technical feature of the present invention advantageously provides for inhibiting Akt nitration preventing glucose uptake and glycolysis in proliferating cells of early PAH. None of the presently known prior references or work has the unique inventive technical feature of the present invention.

Furthermore, the prior references teach away from the present invention. For example, no prior art has utilized combining an antioxidant with a peptide that binds to Akt to prevent oxidation. Furthermore, the inventive technical features of the present invention contributed to a surprising result. For example, it was unexpected that protein oxidation could stimulate the kinase activity of Akt.

The present invention features a targeted conjugated peptide and a composition comprising the targeted conjugated peptide. In preferred embodiments, the conjugated peptide has affinity to or is targeted to Akt and comprises two parts: 1) a peptide portion comprising amino acid sequence Ser-Arg-IIe-Arg-Ser (SRLRS; SEQ ID NO: 1) with affinity for proximity to Tyr 350 residue of Akt; and 2) an antioxidant conjugated to the peptide, wherein the antioxidant including, nitroxide (3-Carboxy-2,2,5,5-tetramethyl-3-pyrroline-1-yloxy), is covalently attached to a free N-terminal amine of the peptide. In preferred embodiments, the peptide that inhibits Akt nitration is conjugated to an antioxidant moiety and the conjugated peptide has affinity to Akt. A non-limiting example of the peptide portion comprises amino acid sequence Ser-Arg-IIe-Arg-Ser (SRLRS; SEQ ID NO: 1) with affinity for proximity to Tyr 350 residue of Akt. A non-limiting example of the antioxidant that is conjugated to the peptide comprises, nitroxide (3-Carboxy-2,2,5,5-tetramethyl-3-pyrroline-1-yloxy), is covalently attached to a free N-terminal amine of the peptide. The conjugated peptide inhibits nitration of Akt.

The present invention also features a method of treating PAH in a patient in need thereof. In preferred embodiments, the method comprises administering an effective amount of a targeted conjugated peptide to the patient to inhibit or prevent Akt nitration. The conjugated peptide comprises two parts: 1) a peptide portion comprising amino acid sequence Ser-Arg-IIe-Arg-Ser (SRLRS; SEQ ID NO: 1) with affinity for proximity to (or targeted to) Tyr 350 residue of Akt; and 2) an antioxidant conjugated to the peptide, wherein the antioxidant including, nitroxide (3-Carboxy-2,2,5,5-tetramethyl-3-pyrroline-1-yloxy), is covalently attached to a free N-terminal amine of the peptide. The conjugated peptide inhibits nitration of Akt.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B shows that Nitroxide conjugated peptide (NP) blocked nitration of Akt. FIG. 1A shows a schematic diagram of nitroxide antioxidant conjugated peptide binding at Tyrosine 350 residue of Akt and inhibition of peroxynitrite radical induced Akt nitration. FIG. 1B shows that SIN-1 (1 mM) for 1 hour significantly increased Y350 nitration of Akt in HPAECs and pretreatment with NP showed a 55% reduction in Akt nitration. Data expressed as Mean±SEM normalized on total protein (stain free), N=3,*p<0.05 versus control, #p<0.05 versus SIN-1.

FIGS. 2A-2C show that Akt nitration is an early event in PAH. FIG. 2A shows immunofluorescence imaging indicating increased Akt nitration in the endothelial and smooth muscle layer of pulmonary arteries of rats after one, two and five weeks of PAH progression (scale bar is 20 μm). FIG. 2B shows Western blot analysis showing significantly increased expression of nitroY350 Akt in sugen treatment groups. Data expressed as Mean±SD, N=6-8*p<0.05 versus control. FIG. 2C shows western blot analysis of healthy controls and IPAH patients and shows significantly increased nitroY350 Akt signal in iPAH. Data expressed as Mean±SD, N=10*p<0.05 versus control.

FIGS. 3A-3E show that NP treatment attenuated Akt nitration in early PAH. FIG. 3A shows a western blot of nitro Akt, phospho Akt and total Akt expression in the lung lysate of sugen two weeks and sugen two weeks plus NP treatment groups. FIG. 3B shows that NP treatment significantly reduced nitro Akt modification. Phosphorylation of Akt at Ser-473 (FIG. 3C) and total Akt expressions (FIG. 3D) were not altered in two-weeks of sugen/hypoxia and NP treatment. FIG. 3E shows that total nitration in lungs was increased in SU2 group and NP treatment did not attenuate the total nitration in the lung significantly, thus, this shows NP selectivity toward Akt nitration. Data expressed as Mean±SD normalized on total proteins, N=6-8*p<0.05 versus control, #p<0.05 versus SU2.

FIGS. 4A-4K show NP controlled hemodynamic and histological alterations. FIG. 4A shows right ventricular systolic pressure (RVSP) was significantly increased in SU2, and NP peptide treatment showed a reduction in RV pressure. FIG. 4B shows Fulton index, the ratio of right to left ventricles plus septum (RV(LV+S)), was significantly increased in SU2 and markedly decreased in NP treatment. FIG. 4C shows correlation analysis between RVSP and Fulton index showed a significant attenuation of PAH phenotype in NP treatment. FIG. 4D shows EVG staining exhibited perivascular fibrosis, vasoconstriction and proliferation in pulmonary arteries of SU2 and these alterations were significantly attenuated with NP treatment (scale is 100 μm). FIG. 4E shows Ki-67 immunohistochemical staining showed increased cellular proliferation in the media of pulmonary artery in SU2 group and NP peptide effectively prevented vascular remodeling and cellular proliferation (scale is 100 μm). Data expressed as Mean±SD, N=6-8*p<0.05 versus control, #p<0.05 versus SU2. FIG. 4F shows the glycolytic rate of HPASMCs, Sin-1 treatment increased glycolysis but NP treatment attenuated this back to control level. ECAR-extracellular acidification rate, DG-deoxy glucose, Data expressed as Mean±SE, N=6-7. Glycolysis (FIG. 4G) and Glycolytic capacity (FIG. 4H) increased with SIN-1 treatment was found attenuated with NP treatment.

FIG. 4I shows mitochondrial oxidative phosphorylation rate decreased with SIN-1 treatment and NP treatment upregulated the oxidative phosphorylation rate to control level in HPASMCs. OCR-oxygen consumption rate FCCP-Trifluoromethoxy carbonylcyanide phenylhydrazone, ROT/AA-Rotenone/Antimycin-A, Data expressed as Mean±SE, N=6-7. NP treatment without affecting any basal respiration rate (FIG. 4J), it significantly attenuated the SIN-1 increased spare respiratory capacity (FIG. 4K) back to control level.

FIGS. 5A-5F show NP treatment balanced glucose influx and glycolysis in PAH. FIG. 5A shows a western blot of total glucose transporter Glut 4 expression in control, SU2 and NP treatment. FIG. 5B shows the ratio of Glut 4 expression in membrane/cytosol was found significantly increased in SU2 and NP treatment controlled Glut 4 translocation. FIGS. 5C and 5D shows HK1 and GAPDH expression increased in sugen/hypoxia treatment represents increased glucose utilization by glycolysis. FIG. 5E shows lactate dehydrogenase (LDH) expression found in SU2 represents increased lactate production in early PAH. FIG. 5F shows pyruvate dehydrogenase expression was found significantly increased in NP treated sugen/hypoxia. Data expressed as Mean±SD normalized on the total protein, N=6-8*p<0.05 versus control, #p<0.05 versus SU2.

FIGS. 6A-6D show NP maintained the glycolytic shift in early PAH. FIG. 6A shows a western blot of phospho GSK3β and GSK3β in SU2 and SU2+NP treatment. Sugen/hypoxia induced increase in GSK3β expression was reversed with NP treatment. FIG. 6B shows decreased G6PD expression in SU2 was upregulated significantly with NP treatment. FIG. 6C shows increased pyruvate carboxylase expression was decreased with NP treatment. FIG. 6D shows mitochondrial biogenesis marker PGC1α expression found increased in SU2 was significantly reduced with NP treatment. Data expressed as Mean±SD, N=6-8*p<0.05 versus control, #p<0.05 versus SU2.

FIG. 6E shows a metabolite analysis of lung lysate showed metabolic reprogramming in SU2 group and attenuation of metabolic changes by NP treatment. Principal component 1 (PC1) and principal component 2 (PC2) plotted that explain 44.4% and 13% of the total variance, respectively. Prediction ellipses for Control, SU2 and SU2+NP groups are shown probability 0.95 that a new observation from the same group will fall inside the ellipse. N=19 data points.

FIG. 7 shows a schematic NP peptide attenuated metabolic reprogramming in pulmonary hypertension. In two-weeks of sugen/hypoxia, increased nitration induced activation of Akt upregulated the glucose transporter Glut 4 to the membrane and this augmented cellular glucose influx. This results in increased aerobic glycolysis and anaplerosis, and a reduction in glycogenesis and pentose phosphate pathway. These metabolic derangements result in increased cellular proliferation and vascular remodeling in lung tissue of early PAH. NP treatment balanced cellular glucose influx, glycogen synthesis, and pentose phosphate pathway. NP corrected the glycolytic shift, improved oxidative phosphorylation and inhibited anaplerotic reprogramming. Thus, proliferative pathological changes in PAH were found to be reversed with NP treatment. PAH— Pulmonary arterial hypertension, NP— nitroxide conjugated peptide, TCA—Tricarboxylic acid cycle, OXPHOS—oxidative phosphorylation.

### DETAILED DESCRIPTION OF THE INVENTION

PAH: Pulmonary arterial hypertension
RV: Right ventricle
eNOS: endothelial nitric oxide synthase
NO: Nitric oxide
TCA: Tricarboxylic acid
ROS: Reactive oxygen species
HIF: Hypoxia-inducible factor
SOD: Superoxide dismutase
NP: Nitroxide peptide
HPAECs: Human pulmonary artery endothelial cells
IPAH: Idiopathic pulmonary arterial hypertension
RVSP: Right ventricular systolic pressure
LV: Left ventricle
S: Septum
EVG: Verhoeffs van gieson
HK1: Hexokinase 1
LDHA: Lactate dehydrogenase-A
GSK: Glycogen synthase kinase
G6PD: Glucose 6-phosphate dehydrogenase
HPASMCs: Human pulmonary artery smooth muscle cells
ECAR: Extracellular acidification rate
OCR: Oxygen consumption rate
GAPDH: Glyceraldehyde 3-phosphate dehydrogenase
PDH: Pyruvate dehydrogenase
PGC-1a: Proliferator-activated receptor gamma coactivator 1-alpha
PPP: Pentose phosphate pathway
PC: Pyruvate carboxylase

As used herein, “administering” and the like refer to the act of physically delivering a composition or other therapy (e.g. a conjugated peptide composition) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

As used herein, the terms “subject” and “patient” are used interchangeably. As used herein, a subject can be an animal (amphibian, reptile, avian, fish, or mammal) such as a non-primate (e.g., cows, pigs, horses, cats, dogs, rats, etc.) or a primate (e.g., monkey, ape and human). In specific embodiments, the subject is a human. In one embodiment, the subject is a mammal (e.g., a human, a dog) having a disease, disorder or condition described herein (i.e., pulmonary arterial hypertension (PAH)). In another embodiment, the subject is a mammal (e.g., a human, a dog) at risk of developing a disease, disorder or condition described herein. In certain instances, the term patient refers to a human under medical care or animals under veterinary care.

As used herein, the terms “health control” or “healthy subject” refers to individuals who had no underlying chronic or acute disease condition.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “effective amount” as used herein refers to the amount of a therapy or medication which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., PAH), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of a conjugated peptide composition described herein is an amount sufficient enough to provide a therapeutic benefit in the treatment or management of pulmonary arterial hypertension (PAH), or to delay or minimize one or more symptoms associated with the presence of the pulmonary arterial hypertension (PAH). A therapeutically effective amount of a composition (conjugated peptide composition) described herein, means an amount of therapeutic agent, alone or in combination with other therapies, which provides a therapeutic benefit in the treatment or management of the diseases described herein. The term “therapeutically effective amount” can encompass an amount that improves overall therapy, reduces or avoids symptoms or causes of the diseases described herein (i.e., pulmonary arterial hypertension (PAH)), or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, and/or other therapies useful in the prevention, management, treatment and/or amelioration of a given disease, disorder or condition known to one of skill in the art such as medical personnel.

Pulmonary arterial hypertension (PAH) is a severe cardiopulmonary disorder instigated by pulmonary vasculature proliferation. Activation of Akt signaling was previously reported to promote vascular remodeling. It was found that the irreversible nitration of Y350 residue in Akt results in its activation. Data of the present invention indicate that Akt nitration is increased in patients with PAH and SU5416/Hypoxia models.

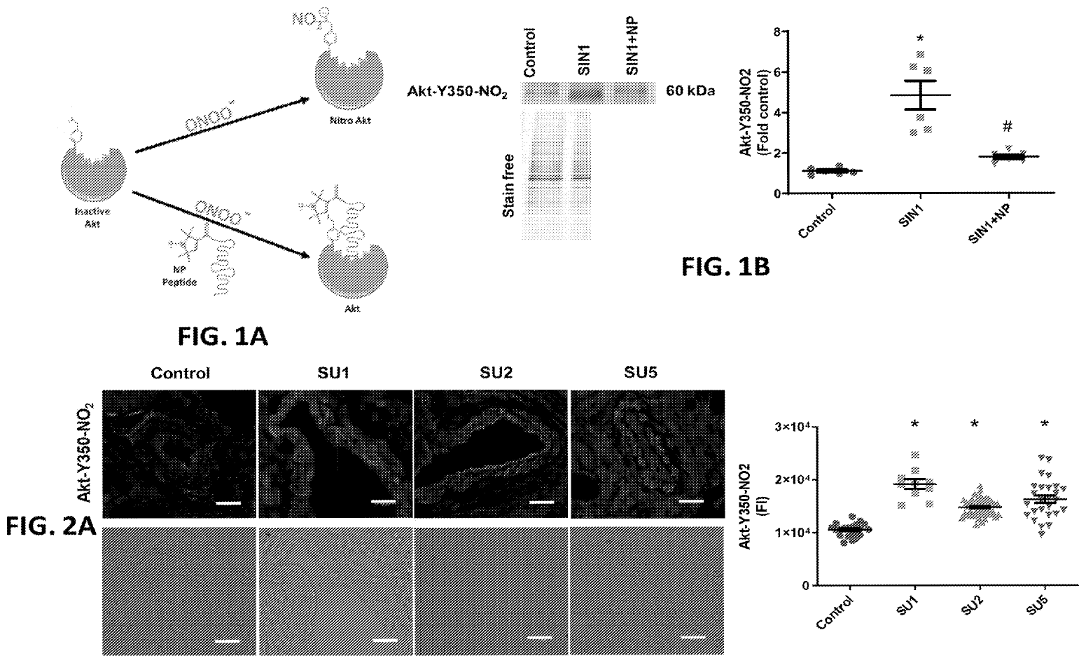

The present invention features a peptide, which has an affinity to Akt, conjugated with an antioxidant moiety—nitroxide (NO). Treatment of SU5416/Hypoxia model with the antioxidant peptide for the first two-weeks significantly reduced nitration of Akt in lungs, attenuated right ventricle (RV) hypertrophy and reduced RV systolic pressure. During the first two-weeks of the disease, Akt nitration induces glycolysis by activation of glucose transporter GLUT4, hexokinase-1, and glyceraldehyde 3-phosphate dehydrogenase enzymes. Decreased glucose 6-phosphate dehydrogenase and increased glycogen synthase kinase 3β shunted glucose via glycolysis in PAH. The increased glycolytic rate upregulated anaplerosis via activation of pyruvate carboxylase. The Akt targeted antioxidant peptide resolved glycolytic switch, activated pentose phosphate, and glycogenesis pathways. Prevention of Akt nitration significantly controlled pyruvate in oxidative phosphorylation by decreased lactate and increased pyruvate dehydrogenases activities. Finally, histopathological studies showed significantly reduced pulmonary vascular proliferation. The present invention shows that preventing Akt nitration using an Akt-targeted antioxidant peptide is a useful treatment option for controlling vascular proliferation in PAHReferring now to FIGS. 1A-7, the present invention features a targeted conjugated peptide with affinity for Akt comprising a peptide conjugated to an antioxidant moiety. The present invention also features composition of the targeted conjugated peptide and method of use of the conjugated peptide for treatment of PAH.

The present invention features a conjugated peptide with affinity for or targeted to Akt comprising a peptide conjugated to an antioxidant moiety, wherein the conjugated peptide prevents or inhibits protein oxidation of the targeted Akt protein.

The present invention also features a conjugated peptide having affinity or targeted to Akt. In some embodiments, the conjugated peptide comprises peptide portion comprising SEQ ID NO: 1 with affinity for proximity to Tyr 350 residue of Akt and an antioxidant conjugated to the peptide. The present invention may also feature a peptide that inhibits Akt nitration, wherein the peptide is conjugated to an antioxidant moiety. In some embodiments, the conjugated peptide has affinity to Akt and comprises a peptide portion comprising SEQ ID NO: 1 with affinity for proximity to Tyr 350 residue of Akt; and an antioxidant conjugated to the peptide. In other embodiments, the conjugated peptide inhibits nitration of Akt

In some embodiments, the conjugated peptide comprises a peptide according to SEQ ID NO: 1 (Ser-Arg-IIe-Arg-Ser (SRLRS). In other embodiments, the conjugated peptide comprises a peptide comprising an amino acid sequence XaaRXaaRXaa (SEQ ID NO: 4), wherein Xaa- is a natural amino acid or a non-natural amino acid. In other embodiments, the peptide has an affinity for proximity to Tyr 350 residue of Akt. In some embodiment, Xaa may be any of the 24 natural amino acids known in the art. In other embodiments, Xaa may be any non-natural amino acids known in the art.

In some embodiments, the antioxidant moiety may comprise any organic compounds with electron acceptor or donor properties, this includes but is not limited to nitroxides, porphyrins, phenols/polyphenols, quinones, flavonoids, vitamins, etc.

In preferred embodiments, a conjugated peptide having affinity to or targeting to Akt comprises two parts: 1) a peptide portion comprising amino acid sequence Ser-Arg-IIe-Arg-Ser (SRLRS; SEQ ID NO: 1) with affinity for proximity to Tyr 350 residue of Akt; and 2) an antioxidant conjugated to the peptide, wherein the antioxidant including, nitroxide (3-Carboxy-2,2,5,5-tetramethyl-3-pyrroline-1-yloxy), is covalently attached to a free N-terminal amine of the peptide. In preferred embodiments, the conjugated peptide inhibits Akt nitration. In other embodiments, the conjugated peptide inhibits nitrations maintaining glucose influx.

In other embodiments, the conjugated peptide inhibits nitration providing a useful treatment option for controlling vascular proliferation in pulmonary arterial hypertension (PAH). In some embodiments, the conjugated peptide inhibits nitration early in development of vascular proliferation of PAH. In some embodiments, the conjugated peptide inhibits nitration providing a useful treatment option for controlling vascular proliferation in pulmonary arterial hypertension (PAH). In further embodiments, conjugated peptide is for the treatment of pulmonary arterial hypertension (PAH). In some embodiments, the conjugated peptide is for the treatment of pulmonary arterial hypertension (PAH). In other embodiments, the conjugated peptide is for the treatment of proliferative disorders including cancer, atherosclerosis, rheumatoid arthritis, psoriasis, idiopathic pulmonary fibrosis, asthma, restenosis, or diabetic retinopathy.

The present invention features a composition comprising a targeted conjugated peptide with affinity for or targeting to Akt. In some embodiments, the targeted conjugated peptide comprises a peptide conjugated to an antioxidant moiety. In preferred embodiments, the composition has affinity to Akt and comprises a conjugated peptide comprising two parts: 1) a peptide portion comprising amino acid sequence Ser-Arg-IIe-Arg-Ser (SRLRS; SEQ ID NO: 1) with affinity for proximity to Tyr 350 residue of Akt; and 2) an antioxidant conjugated to the peptide, wherein the antioxidant including, nitroxide (3-Carboxy-2,2,5,5-tetramethyl-3-pyrroline-1-yloxy), is covalently attached to a free N-terminal amine of the peptide. In preferred embodiments, this composition inhibits nitration of Akt.

The present invention also features a composition having affinity to or targeted to Akt. In some embodiments, the composition comprises a conjugated peptide comprising two parts a peptide portion comprising SEQ ID NO: 1 with affinity for proximity to Tyr 350 residue of Akt; and an antioxidant conjugated to the peptide, wherein the antioxidant is nitroxide (3-Carboxy-2,2,5,5-tetramethyl-3-pyrroline-1-yloxy), is covalently attached to a free N-terminal amine of the peptide. The present invention may also feature a composition comprising a peptide that inhibits Akt nitration, wherein the peptide is conjugated to an antioxidant moiety. In some embodiments, the 20. conjugated peptide has affinity or targeted to Akt and comprises two parts a peptide portion comprising SEQ ID NO: 1 with affinity for proximity to Tyr 350 residue of Akt; and an antioxidant conjugated to the peptide.

The present invention further features a method of treating pulmonary arterial hypertension (PAH) in a patient in need thereof, the method comprising administering an effective amount of a conjugated peptide to the patient to inhibit or prevent Akt nitration, the conjugated peptide comprises two parts: 1) a peptide portion comprising amino acid sequence Ser-Arg-IIe-Arg-Ser (SRLRS; SEQ ID NO: 1) with affinity for proximity to Tyr 350 residue of Akt; and 2) an antioxidant conjugated to the peptide, wherein the antioxidant including, nitroxide (3-Carboxy-2,2,5,5-tetramethyl-3-pyrroline-1-yloxy), is covalently attached to a free N-terminal amine of the peptide,

In some embodiments, the conjugated peptide inhibits Akt nitration maintaining glucose influx. In other embodiments, the conjugated peptide inhibits nitration early in development of vascular proliferation of PAH. In further embodiments, the conjugated peptide inhibits nitration providing a useful treatment option for controlling vascular proliferation in PAH. Preferably, the conjugated peptide inhibits nitration early in development of vascular proliferation of PAH and is a treatment for PAH. In some embodiments, the conjugated peptide reverses the metabolic reprogramming from glycolysis to oxidative phosphorylation and prevents cellular proliferative changes. In other embodiments, the conjugated peptide protects proteins from oxidation.

In other embodiments, the conjugated peptide of claim 1, wherein the conjugated peptide is for the treatment of proliferative disorders including cancer, atherosclerosis, rheumatoid arthritis, psoriasis, idiopathic pulmonary fibrosis, asthma, restenosis, diabetic retinopathy.

In certain circumstances, the present invention addresses a significant need for PAH as few alternative effective therapies currently available. In addition, the targeted selectivity for Atk1 nitration (i.e. will not affect Atk1 phosphorylation) and will not affect other physiological processes controlled by Atk1 signaling pathways)

### EXAMPLE

The following are non-limiting examples of the present invention. Material and methods that supported these examples are described below. It is to be understood that said example is not intended to limit the present invention in any way. Equivalents or substitutes are within the scope of the present invention.

### Materials and Methods

### Development of Nitroxide Conjugated Affinity Peptide

Nitroxide peptide (NP) was synthesized by 21st CenturyBiochemicals company (Marlboro, Mass.) from two parts, one being the peptide part with the affinity to Akt near Tyr 350 residue (Ser-Arg-IIe-Arg-Ser, SEQ ID NO: 1)—and the other, a conjugated antioxidant—nitroxide (3-Carboxy-2,2,5,5-tetramethyl pyrroline-1-yloxy) covalently attached to the free N-terminal amine. The peptide was purified and used immediately after preparation of the solution.

### Sin-1 Induced Akt Nitration Assay

Human pulmonary artery endothelial cells (HPAECs) were cultured using an endothelium media specific for HPAEC with 10% FBS. Cells were used from passages 3-6. All experiments were performed on 80-90% confluent cells. Akt nitration was induced by treatment with 3-Morpholinosyndnomine (SIN-1) (1 mM) for 1 hour. NP pretreatment groups were treated with 1 μM NP for 30 min, and then cells were washed in PBS and treated with SIN-1 for 1 hour. All treatments were accompanied by including controls with the corresponding vehicle. After treatment, the cells were washed in PBS and lysed in a RIPA buffer containing protease and phosphatase inhibitor cocktail.

### Human Subjects

De-identified lung samples consisted of patients with a diagnosis of Group I PAH—Idiopathic pulmonary arterial hypertension (IPAH) group, N=10) and healthy controls (Control group, N=10) were obtained through Pulmonary Hypertension Breakthrough Initiative (PHBI). The PHBI study protocol was approved by the Institutional Review Boards of the participating sites in the network, and all sites were adherent to the requirements of the U.S. Federal Policy for the Protection of Human Subjects (45 CFR, Part 46), and supported the general ethical principles of the Declaration of Helsinki.

### Rat Model of PH

Female Sprague Dawley rats (200-250 g) obtained from Charles River (Wilmington, Mass., USA) were used in this study. Animals were housed at 22° C., 12-h light: dark cycle, and had free access to standard rodent food and water ad libitum. All experimental procedures were approved by the University of Arizona Institutional Animal Care and Use Committee. Generally, PAH was induced by a single injection of SU5416 (50 mg/kg) subcutaneously, followed by 3 weeks of hypoxia (10% 02) and 2 weeks of normoxia. This study included five animal groups: control group; SU1, rats were analyzed after 1 week of SU5416 and hypoxia treatment; SU2, rats were analyzed after 2 weeks of SU5416 and hypoxia treatment; SUS, rats were analyzed after 5 weeks of SU5416 treatment (3 weeks of hypoxia with a following 2 weeks of normoxia); and SU2+NP peptide treatment 0.1 mg/kg/day intravenous (i.v.) injection started with SU5416 treatment for two-weeks.

### Hemodynamic Measurement

Animals were anesthetized with inactin 100 mg/kg intraperitoneally (i.p.) (T133, Sigma-Aldrich). A customized pressure transducer catheter (SPR-513, Millar Instruments, Houston, Tex.) was inserted into RV via the right jugular vein and advanced into the right ventricle to monitor right ventricular systolic pressure (RVSP) as described previously. Briefly, the pressure transducer catheter was connected to a Millar Transducer Control Unit TC-510 and PL3504 PowerLab 4/35 data acquisition system (AD Instruments, Colorado Springs, Colo.) to monitor RV pressure for 30-min. After this a tracheal catheter was connected to a ventilator system (Harvard Rodent Ventilator-683; Harvard Apparatus, South Natick, Mass.), the thorax was opened, and the lungs flushed with 0.9% sodium chloride through the right ventricle (RV). Heart and lungs were collected from animals; the RV free wall was separated from the left ventricle (LV), and the septum (5). Fulton index (RV/LV+S ratio) as a parameter of RV hypertrophy was calculated. The total wet lung weight was measured and normalized by the body weight of the animal. The left lung was fixed in formalin and embedded in paraffin for histological examination. The other portion of the lung was stored at −80° C. for further biochemical studies.

### Histological Analysis

For the morphometric assessment of pulmonary vessels 5-μm tissue sections were dewaxed and stained with elastic fibers—Verhoeffs van gieson (EVG) by HistoWiz Inc. (Brooklyn, N.Y.) using standard operating procedures and a fully automated workflow system. Ten transversely sectioned pulmonary arteries (diameter<300 μm) within each category per each animal (N=6 per group) were randomly selected from the whole-slide 40X digitized image Aperio AT2 scanner (Leica Biosystems, Ill.). Immunohistochemistry was performed using standard protocols. The sections were deparaffinized and incubated with primary antibodies against Akt Y350 NO2 (1:100), and Ki67 (1:800) (ab15580 Abcam); Dab Rabbit H1 (pH 6) for 20 min. The morphometric analysis was done by an investigator by a blinded grouping fashion. The wall thickness of the pulmonary artery (PA) was measured using the publicly available software Fiji is just ImageJ (http://fiji.sc/Fiji; in the public domain).

### Western Blot Analysis

For an analysis of the total lung protein, lung tissues were lysed as previously described. Briefly, 20-40 mg of lung tissue (human/rat) was lysed in Permeabilization Buffer mixed with protease and phosphatase inhibitor cocktails using a Fisher Homogenizer 850. The homogenate was centrifuged at 10,000 g for 10 min, and the supernatant was carefully collected. Cell membrane and cytosolic fractions were isolated using FractionPREP™ cell fraction kit (Biovision, Milpitas, Calif.) according to manufacturer's instruction. The protein concentration was measured using the BCA protein assay kit (Thermo Scientific, Rockford, Ill.). Samples were incubated with 6×Laemmli sample buffer (Boston Bioproducts Inc., Ashland, Mass.), for 5 min at 95° C., loaded on the 4-20% SDS-PAGE Mini-PROTEAN TGX Stain-Free™ gels (Bio-Rad Laboratories Inc., Hercules, Calif.) and separated by electrophoresis. Protein bands were transferred using Trans-Blot Turbo transferring system (Bio-Rad Laboratories Inc.) and then blocked with 5% bovine serum albumin in Tris-buffered saline. Membranes were probed using antibodies against Glut 4 (07-1404) from Millipore, pyruvate carboxylase (PC) (Ab229267) & peroxisome proliferator-activated receptor gamma coactivator 1-alpha (PGC-1α) (Ab191838) from Abcam, pyruvate dehydrogenase (PDH) (45-6600) from Invitrogen, phospho Akt (Ser 473) (4060S), Akt (9272S), hexokinase 1 (HK1) (C35C4), glyceraldehyde-3 phosphate dehydrogenase (GAPDH) (2118S), lactate dehydrogenase-A (LDHA) (2012S), phospho glycogen synthase kinase (GSK) 3 β (Ser9) (9336S), GSK3 β(9315S) and glucose 6-phosphate dehydrogenase (G6PD) (8866S) from Cell Signaling Technology. Mouse monoclonal anti-nitrotyrosine antibodies were obtained from Calbiochem, La Jolla, Calif. (487923). Akt Y350 NO2 antibody obtained as a custom-made antibody against Akt1-nitro (341-353) and the sequence was CGRLPF-nitroY-NQDHEK (SEQ ID NO: 2). Two peptides were developed with and without Y350 nitration. The antibody developed against nitrated peptide cleaned using non-nitrated peptide (Akt1 (341-353): CGRLPFYNQDHEK, SEQ ID NO: 3) to remove nonspecific antibodies. Affinity purification was done using large reusable affinity purification columns against the unmodified and nitrated peptides. ELISA validation showed specificity towards the nitrated sequence and therefore selectively purified antibodies raised to nitro-Akt (Pacific Immunology Corp. Ramona, Calif.). Since the quantification of the Akt nitration is not fully established by mass spectrometry, a common method was used by newly developed antibodies for the quantification of specific Y350 nitration of Akt. The reactive bands were visualized by chemiluminescent ChemiDoc™ MP Imaging System (Bio-Rad Laboratories Inc., Hercules, Calif.) and analyzed using Image Lab™ software. The protein loading was normalized per total sample protein as fold control using stain free gels as previously described. This normalization is equal to housekeeping genes normalization and rigorously evaluated by Bio-Rad company (http://www.bio-rad.com/en-us/applications-technologies/stain-free-imaging-technology?ID=NZ0G1815) and others in comparison with beta-actin normalization.

### Seahorse Assays

For the glycostress Seahorse assay, human pulmonary artery smooth muscle cells (HPASMCs) were seeded in a 24 well Seahorse cell culture microplate at 50000/well and kept overnight to form a monolayer. On the day of experiment, the media was aspirated and cells were incubated at 37° C. in a non-CO2 incubator for 1 hour with 0.5 mL XF base medium (cat #102353-100, Agilent, Santa Clara, Calif.) supplemented with 2 mM glutamine. Glucose (56 μL, 100 mM), Oligomycin (62 μL, 100 μM) and 2-deoxyglucose (69 μL, 500 mM) was added to the flux pack wells (cat #102342-100, Agilent, Santa Clara, Calif.). ECAR (extracellular acidification rate) was then measured using Seahorse Bioscience XFe24 extracellular flux analyzer (Agilent, Santa Clara, Calif.) according to the manufacturer's protocol. For the mitostress assay, cells were prepared like the glycostress assay with the exception that the XF base incubation media was supplemented with pyruvate (1 mM), glutamine (2 mM) and glucose (10 mM). Oligomycin (56 μL, 10 μM), FCCP (62 μL, 10 μM) and Rotenone+Antimycin-A (69 μL, 5 μM) were added to the flux pack wells. OCR (oxygen consumption rate) was then identified according to the manufacturer's protocol. Data was normalized to cell number as determined by flow cytometry. All the chemicals used for these experiments, unless specified, were procured from Sigma-Aldrich.

### Lactate Dehydrogenase Assay

LDH activity in lung tissue was assessed by Lactate Dehydrogenase Activity Colorimetric Assay Kit (K726, BioVision Inc. Calif., USA) according to manufacturer's instruction. All values were normalized with respect to protein concentrations as determined by the Pierce™ BCA Protein Assay Kit (Thermo Scientific, Rockford, Ill.).

### Pyruvate Dehydrogenase Assay

Lung tissue was lysed using RIPA buffer and the lysate was used for the PDH activity. PDH activity was measured with PDH Activity Assay Kit (MAK183, Sigma-Aldrich) per the manufacturer protocol. All values were normalized with respect to protein concentrations as determined by the Pierce™ BCA Protein Assay Kit.

### Metabolic Intermediates Analysis

Data were acquired using the West Coast Metabolomics Center at UC Davis. Briefly, Restek corporation Rtx-5Sil MS column was used with helium mobile phase at temperature interval (50-330° C.) and flow-rate: 1 mL min-1. Injection volume was 0.5 μL at 50° C. ramped to 250° C. by 12° C. s-1. Oven temperature started at 50° C. for 1 min, then ramped at 20° C. min-1 to 330° C., and held constant for 5 min. Mass spectrometry analysis was done on a Leco Pegasus IV mass spectrometer with unit mass resolution at 17 spectra s-1 from 80-500 Da at −70 eV ionization energy and 1800 V detector voltage with a 230° C. transfer line and a 250° C. ion source. Raw data files were preprocessed directly after data acquisition and stored as ChromaTOF-specific*.peg files, as generic*.txt result files and additionally as generic ANDI MS*.cdf files. ChromaTOF vs. 2.32 was used for data preprocessing without smoothing, 3s peak width, baseline subtraction just above the noise level, and automatic mass spectral deconvolution and peak detection at signal/noise levels of 5:1 throughout the chromatogram. Apex masses were reported for use in the BinBase algorithm. Result*.txt files were exported to a data server with absolute spectra intensities and further processed by a filtering algorithm implemented in the metabolomics BinBase database. Significantly changed metabolites were processed with principal component analysis (PCA).

### Statistical Analysis

Statistical analysis was performed using GraphPad Prism version 5.01 (GraphPad Software, San Diego, Calif.). Outliers were identified with Grubbs' test using GraphPad outlier calculator (Alpha=0.5) at https://www.graphpad.com/quickcalcs/Grubbs1.cfm. The mean value (±SE) was calculated for all samples, and significance was determined by the unpaired t-test, Mann-Whitney U t-test or analysis of variance (ANOVA). For ANOVA, Bonferroni multiple comparison tests to compare the selected pairs of columns were used. The significance is calculated using a 95% confidence interval.

### Results

### Example 1: Inhibition of Akt Nitration by NP Peptide

In the current investigation, the Sugen/hypoxia PAH model was treated with the newly designed defensive peptide, which blocks the Akt nitration site at Y350. This peptide has two parts, one being the peptide part that has an affinity to Akt (Ser-ARg-IIe-ARg-Ser or SRLRS, SEQ ID NO: 1)—and the other, a conjugated antioxidant—nitroxide. FIG. 1A depicts the functioning of the defensive peptide; the peptide part specifically binds near to the Tyr-350 residue of Akt and masks it from nitration. The nitroxide antioxidant part conjugated to the affinity peptide would help scavenge free radicals from tyrosine residue, and thereby prevent Akt modifications by peroxynitrite. The efficiency of the developed defensive nitroxide-peptide was tested by treating HPAECs. Initially, HPAECs were treated with a peroxynitrite donor, SIN-1, which increased the Akt nitration. The amount of Y350 Akt nitration was recognized by using the custom-made antibody specific towards the nitrated Y350 sequence of Akt, which showed a very clear increase in Akt nitration in SIN1 treated cells (FIG. 1B). NP peptide was incubated with HPAECs for 30 min then it was washed with PBS and finally SIN-1 was added to cells. Interestingly, it was found that NP treatment showed 55% reduction of Akt nitration in HPAECs (FIG. 1B). Also, this additionally demonstrates the specificity of the antibody towards recognition of nitro-Y350 Akt as well as efficacy of NP in blocking the nitration of Akt at the Y350 residue in pulmonary endothelial cells. Antibodies are commonly used for quantification of post-translational modification since mass spectrometry analysis is not a quantitative method especially for nitration modification of proteins.

### Example 2: Akt Nitration an Early Event Initiating PAH

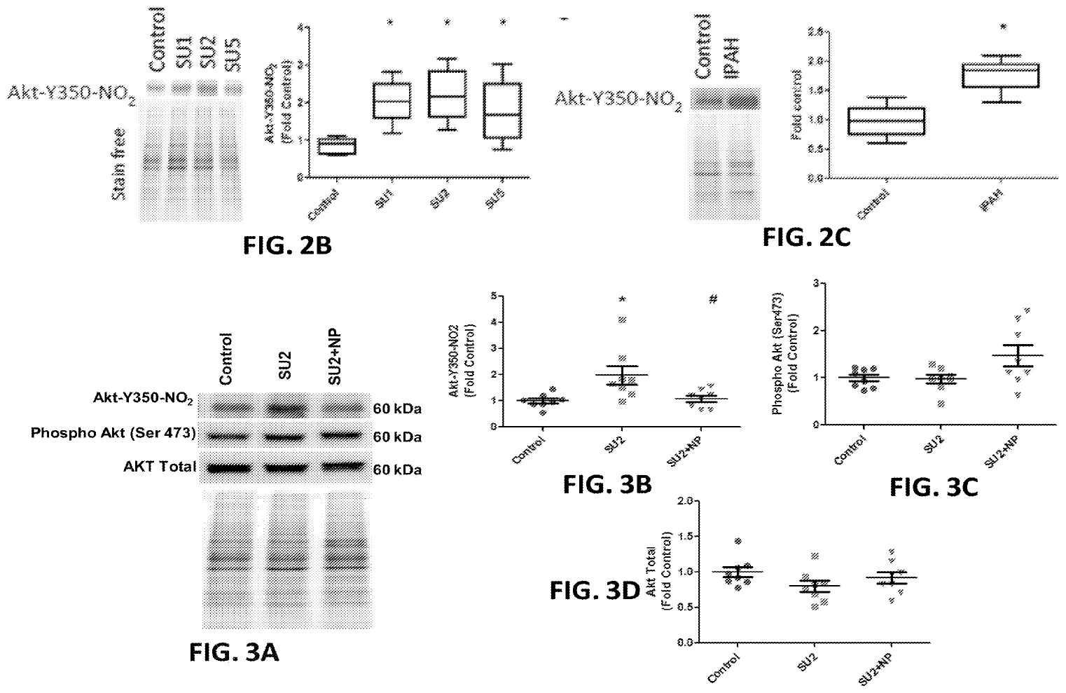

In the present invention, the onset of Akt nitration was screened in three-time phases after sugen injection, after one week (early), two-weeks, and finally at five weeks. Interestingly, in immunofluorescence imaging, increased nitration of Akt was found in endothelial and smooth muscle layers of pulmonary arteries. This was observed as early as week one of the disease induction and trails in two and five weeks of the disease (FIG. 2A). This observation was also correlated with increased nitro Akt protein modification in lung lysates, in sugen one, two, and five weeks of PAH model's progression (FIG. 2B). This observation signifies that the nitration of Akt starts early in the pathogenesis of PAH. Importantly, screening of human lung tissue samples from the PHBI cohort also revealed increased Akt nitration compared with healthy controls (FIG. 2C).

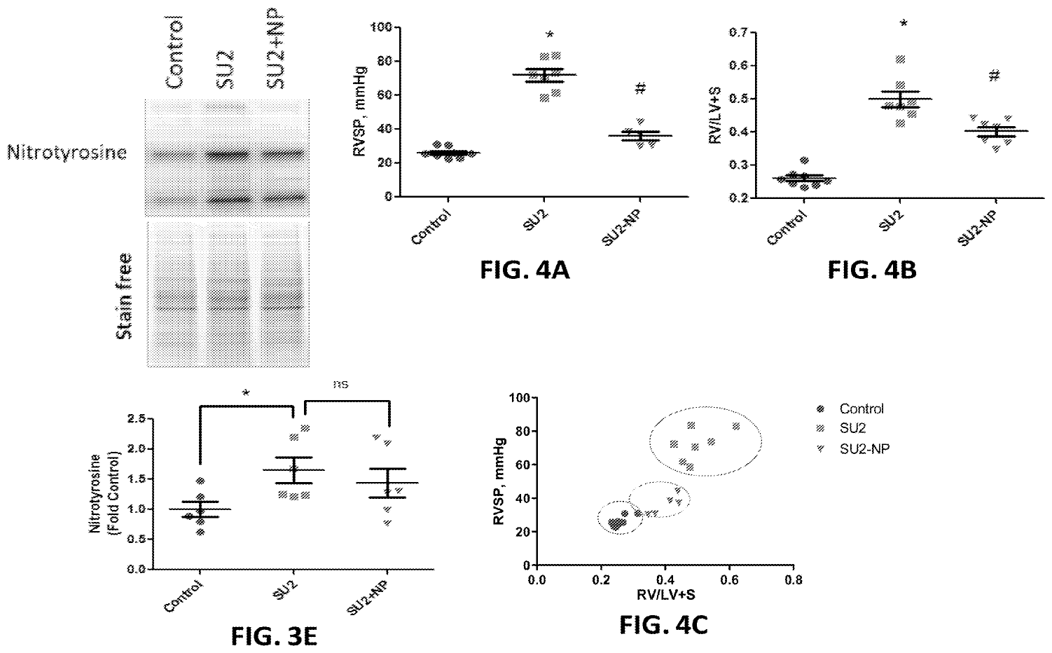

Based on these findings, sugen/hypoxia rats were treated with NP peptide 0.1 mg/kg/day i.v. for two-weeks. The NP treatment showed a remarkable reduction of nitroAkt modification in the lung tissue (FIG. 3A). Here, the NP peptide significantly blocked Akt nitration at the inception of PAH development (FIG. 3B). Phosphorylation of Akt at Ser-473, an important triggering factor in the PAH pathogenesis was found not altered in two-weeks of sugen/hypoxia. Also, the NP treatment did not change the total Akt expression (FIGS. 3C & 3D). These results suggest that the nitration of Akt is a primary episode in PAH pathogenesis and masking the nitration of Akt with NP does not affect the phosphorylation levels or the total expression of Akt. Importantly, total lung nitration levels were increased in SU2 group, and treatment with NP did not significantly alter total nitration, confirming specificity toward Akt nitration (FIG. 3E).

### Example 3: NP Attenuated Ventricular Pressure and Histological Changes

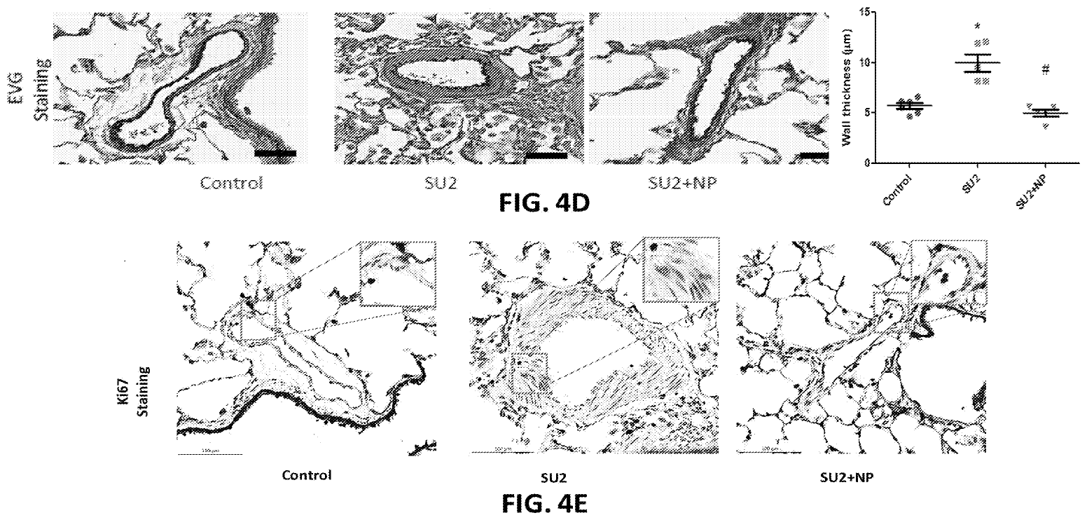

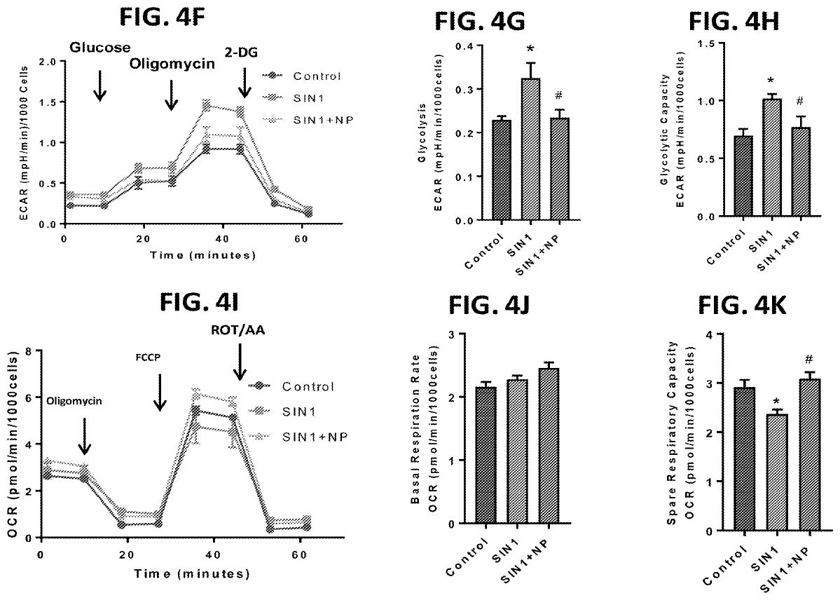

RV function in PAH was screened in two-weeks of sugen/hypoxia with NP treatment. Hemodynamic measurement by RV catheterization indicated an increase in RVSP in two-weeks of sugen/hypoxia PAH model. The NP peptide treatment showed a reduction in RV pressure from 71.9±3.6 to 36.1±2.6, p<0.001 (FIG. 4A). In accordance with increased pressure, the RV of two-week PAH showed an increase in Fulton index—the hypertrophic changes (FIG. 4B), and this was found reversed with NP treatment. FIG. 4C shows a significant correlation between RVSP and Fulton index in SU/hypoxia model and early NP intervention reversed both remodeling and heart hypertrophy back to almost control levels.

The histological alterations were studied in lung tissue associated with increased RV pressure in two-weeks of sugen/hypoxic PAH. EVG staining showed perivascular fibrosis, vasoconstriction, and proliferation in pulmonary arteries. This signifies vascular obstruction and increased RV pressure overload in the pulmonary artery (FIG. 4D). Ki-67 immunohistochemical staining—a crucial protein marker for cellular proliferation was found increased in smooth muscle cells of the pulmonary artery in the rat PAH model. NP peptide effectively prevented vascular remodeling and cellular proliferation in PAH (FIG. 4E). These observations suggest that NP peptide reduced proliferative pathological changes and maintained vascular histology, resulting in reduced RV pressure and thereby prevented PAH development.

Concurrent to proliferative phenotypical changes in smooth muscle cells of pulmonary artery, increased glycolysis and decreased oxidative phosphorylation were observed in HPASMCs treated with peroxynitrite donor, SIN-1. SIN-1 treatment enhanced the overall glycolysis and glycolytic capacity in HPASMC as demonstrated in FIGS. 4F-4H. NP treatment attenuated the nitration induced glycolysis and brought back to control level by inhibiting Akt nitration. Moreover, SIN-1 treatment attenuated the mitochondrial respiration but on the other hand, NP treatment reversed the SIN-1 induced changes in mitochondrial respiration by significantly enhancing mitochondrial function as demonstrated by spare respiratory capacity, without affecting the basal respiration rate (FIGS. 4I-4K). These observations explain a concise metabolic transition in smooth muscle cells from oxidative phosphorylation to glycolysis, similar to highly proliferative cancer cells.

### Example 5: NP Treatment Controlled Glucose Uptake and Glycolysis in PAH

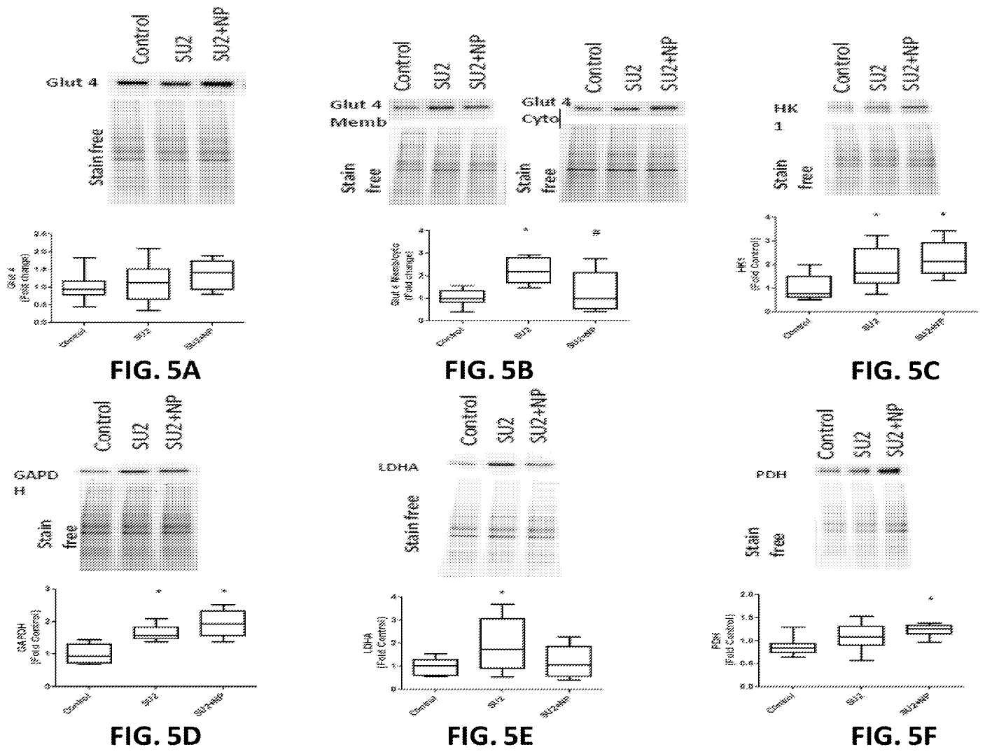

In the early sugen/hypoxic PAH model, glycolytic metabolism was examined, usually found altered in highly proliferative cells. Glucose uptake into the cell is controlled by glucose transporters, and Glut4 is known to be Akt dependent. Akt is an important regulator of Glut4 translocation to the cell membrane. During the first two-weeks of the disease, lungs demonstrated the same Glut4 expression, but increased Glut4 localization was identified in membrane fraction. This correlated with the observed increase in Akt nitration and NP treatment attenuated Glut4 translocation to membrane (FIG. 5A, 5B). It could therefore be inferred that Glut4 translocation and subsequent increase in glucose uptake was increased due to Akt nitration. The increased glucose concentration in the cytosol and activation of the Akt pathway trigger high glycolysis rate by upregulating HK1 and GAPDH enzymes (FIG. 5C, 5D). In early PAH, increased glycolysis was found to result in increased production of the aerobic product, lactate from pyruvate, by the enzyme LDHA. SU2 lung tissue showed increased LDHA expression and activity (FIGS. 5E, 5F). Prevention of Akt nitration significantly reduced pyruvate disposal to lactate by attenuating LDHA expression and its activity. This resulted in an increased expression of PDH enzyme and its activity to normalize the glycolytic metabolism through oxidative phosphorylation. Downregulation of LDHA decreased acidification rate by lactate and upregulation of PDH helps to supply increased acetyl Co-A for oxidative phosphorylation in the mitochondria. This explains the Seahorse analysis data in FIGS. 4F and 4I. This substantiates that NP treatment potentially balances the glycolytic shift in pulmonary vascular cells of PAH favoring flux of pyruvate into oxidative phosphorylation restoring metabolically reprogrammed cells.

### Example 6: Pentose Phosphate Pathway and Glycogenesis Decreased in PAH

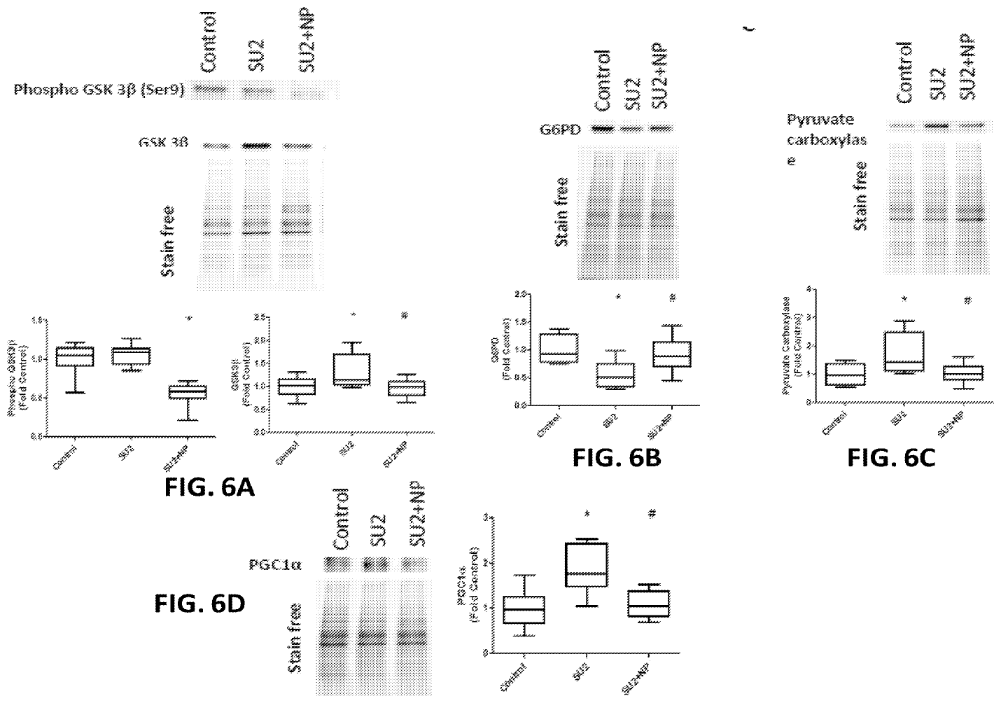

In accordance with increased glycolysis, the compensatory glycogenesis, the anabolic pentose phosphate pathway (PPP) and PC mediated anaplerosis pathways were also screened in two-weeks of sugen/hypoxia PAH. GSK3β which downregulates glycogen synthesis by inhibiting glycogen synthase, was found increased in sugen/hypoxia. Glucose shuttling in PPP from glucose 6-phosphate is driven by G6PD and was found decreased in two-weeks of PAH (FIGS. 6A, 6B). Interestingly, the inhibition of Akt nitration with NP effectively maintained glycogen synthesis and PPP. The increased glycolytic rate in sugen/hypoxia resulted in the activation of PC, increasing oxaloacetate from pyruvate going to TCA cycle; thus, upregulating anaplerosis (FIG. 6C). Increased anaplerosis may affect pulmonary vascular cell proliferation in PAH. The NP peptide treatment during the first two-weeks at 0.1 mg/kg/day i.v. resolved anaplerosis by decreasing the expression of PC. Altered oxidative phosphorylation in PAH could result largely in mitochondrial dysfunction. Two-weeks sugen/hypoxia treatment increased PGC-1α expression, a major transcriptional coactivator and a chief compensatory controller of mitochondrial biogenesis in hypoxic stress and cellular proliferation. Importantly, prevention of Akt nitration significantly controlled PGC-1a expression in the lung tissue (FIG. 6D). Lung metabolite profiling was performed using LC-MS quantitative proteomics for Control, SU2 and SU+NP groups. Significantly changed (p<0.05) metabolites were analyzed with principal component analysis and the data showed a significant separation between all groups. In FIG. 6E, control and SU2 groups showed further separation by PCA. NP treatment results in moving diseased animals toward control that can indicate resolution of reprogramed metabolism in the PAH model. Indeed, 30 metabolites were significantly restored by NP treatment.

### Example 7: NP Peptide Attenuates Metabolic Reprogramming in Pulmonary Hypertension

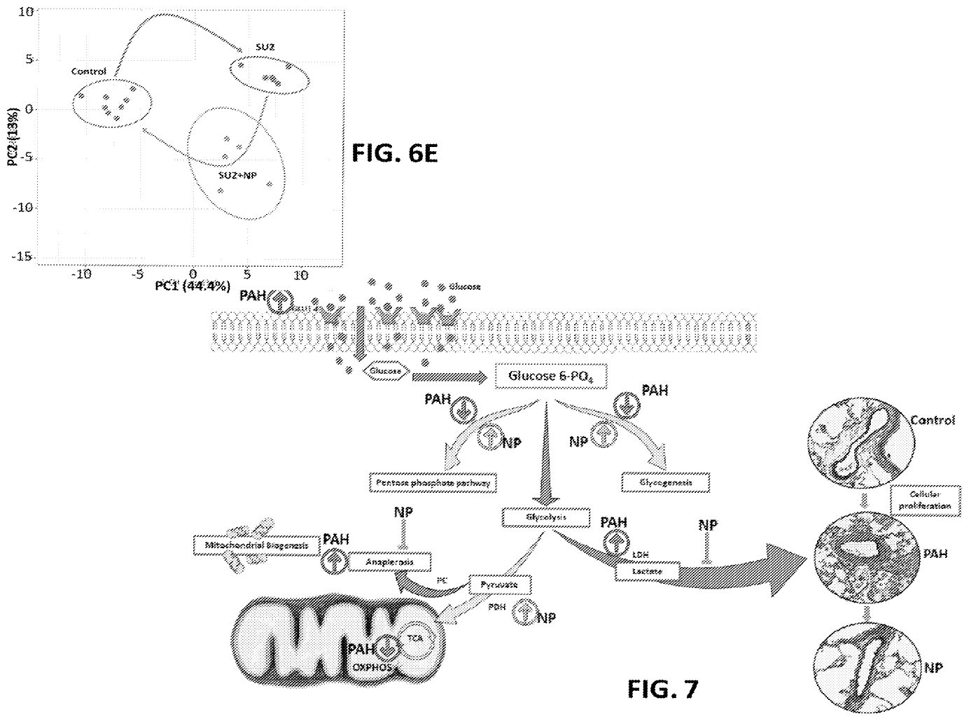

In two-weeks of sugen/hypoxia, increased nitration induced activation of Akt upregulated the glucose transporter Glut 4 to the membrane and this augmented cellular glucose influx. This results in increased aerobic glycolysis and anaplerosis, and a reduction in glycogenesis and pentose phosphate pathway. These metabolic derangements result in increased cellular proliferation and vascular remodeling in lung tissue of early PAH. NP treatment balanced cellular glucose influx, glycogen synthesis, and pentose phosphate pathway. NP corrected the glycolytic shift, improved oxidative phosphorylation and inhibited anaplerotic reprogramming. Thus, proliferative pathological changes in PAH were found to be reversed with NP treatment. PAH— Pulmonary arterial hypertension, NP— nitroxide conjugated peptide, TCA—Tricarboxylic acid cycle, OXPHOS— oxidative phosphorylation (FIG. 7).

### DISCUSSION

Pulmonary arterial hypertension (PAH) is a progressive and fatal condition characterized by narrowing of the pulmonary arteries because of muscular thickening and intimal proliferation. Abnormal Akt activation causes normal cells to undergo proliferative conversion, but in PAH, the cause and activation of Akt are not fully elucidated. The nitration of Akt plays a crucial role in its activation, and it was found that increased Akt nitration in patients with PAH as well as in sugen/hypoxia model. Akt nitration can activate eNOS phosphorylation, and this, in turn, increases peroxynitrite formation, nitrosative stress, and mitochondrial dysfunction. Prolonged mitochondrial dysfunction is responsible for PAH development with vascular damage and metabolic disorder. The study of metabolic alterations can be performed either by metabolomics or proteomics analysis.

In the present invention, protein expression was followed as well as metabolites profile were compared between groups. This approach clearly demonstrated PAH specific alterations in the metabolic enzymes and metabolites, unveiling the protective mechanism of the antioxidant peptide in the context of Akt nitration. Interestingly, during the two-week sugen/hypoxic model, activation of Akt upregulated glucose transporter, Glut 4, to the membrane and this augmented cellular glucose influx. This results in increased aerobic glycolysis and anaplerosis, and a reduction in glycogenesis and PPP, similar to highly proliferative cancer cells.

Histological studies identified cellular proliferation and vascular remodeling in lung tissue of early PAH. These pathological modifications recapitulate increased RV systolic pressure and Fulton index in early PAH. The NP peptide inhibited Akt nitration at Tyr-350 and controlled metabolic derangement in early PAH. NP treatment balanced cellular glucose influx, glycogen synthesis, and PPP. These events controlled glycolysis, oxidative phosphorylation, and inhibited anaplerotic reprogramming. Thus with NP treatment, proliferative pathological changes in PAH were found to be reversed, and RV functioning improved with normal RVSP. The summary of observed metabolic alterations and effect of NP treatment is combined in FIG. 7.

As the incidence of PAH is high in females, female rats were used to examine the anaplerotic reprogramming and Akt nitration events in PAH. The high incidence of PAH in females could perhaps be due to increased nitric oxide production and nitrosative stress development. NO, and superoxide anion produced in hypoxic stress potentiates a variety of biological processes including peroxynitrite formation, nitration of many tyrosine residues of proteins and a decrease in antioxidant status. Recently, it was unveiled that Akt1 nitration at Tyr-350 residue enhances the phosphorylation of eNOS at Ser617/1179 and its translocation to mitochondria in pulmonary artery endothelial cells. Excitingly, in the present invention, using the newly developed nitro Akt (Y350-NO2) antibody, it was discovered that nitration of Akt at Tyr-350 residue is an early molecular modification possibly responsible for the pathogenesis and progression of PAH. Immunohistochemistry and protein expression studies authenticated that Akt nitration starts early from one week of the disease and trails up in two and five weeks of the sugen/hypoxic model of PAH. Increased oxidative and nitrosative stress in early sugen/hypoxic PAH could have potentiated these nitration events. Pathological instigation of the Akt pathway has been described in many cancers, specifically in lung carcinogenesis, nitrosative stress induced tyrosine nitration potentiated cellular proliferation and metastasis.

The present invention features an antioxidant NP peptide having two moieties; one a peptide part, designed complimentary to the Tyr-350 residue of Akt to obtain affinity and the second, an antioxidant nitroxide that helps to scavenge free radicals/electrons. Nitroxide is a stable free radical, containing a nitroxyl group and an unpaired electron. This can easily diffuse through the cell membranes. In biological settings, they demonstrate SOD and catalase like activities, preventing the Fenton and Haber-Weiss reactions and mitigating protein and lipid damage. The efficacy of the antioxidant peptide (NP) was screened in HPAECs by inducing nitrosative stress with the peroxynitrite donor SIN-1. Extracellular treatment with NP alleviated (55%) nitration of Akt in endothelial cells. The dose of NP peptide used in this study is 100 times lower than the concentration of the antioxidant, TEMPOL, normally used in treating PAH and it does not affect the redox homeostasis of the cellular environment. It was also recently reported that TEMPOL treatment was not effective in preventing pulmonary arterial remodeling, and in fact, increased the severity of PAH.

In accordance with the initial observation, Akt nitration was found to be an early event, and NP was tested in sugen/hypoxic PAH model at week 2 of disease progression. Interestingly, it was found that the NP peptide was effective in blocking the nitration of Akt in early PAH. In late PAH, activation of Akt at Ser-473 plays a crucial role in the reprogramming of metabolic pathways and PAH pathogenesis. But intriguingly in early PAH, there was neither any phosphorylation at Ser-473 of Akt nor any change in the expression of total Akt. This observation strengthens the idea that the activation of Akt occurring in early PAH might be through nitration of Akt. Akt activation is strongly associated with cellular proliferation and vascular remodeling. These results suggest that the inhibition of Akt nitration with NP peptide could mitigate early Akt activation and vascular modification.

Pulmonary vascular remodeling is among the major factors in initiating higher pulmonary vascular resistance and pulmonary arterial pressure. Akt plays a crucial role in hypoxia-induced pulmonary vascular remodeling. In early sugen/hypoxic PAH models, increased RV systolic pressure and right ventricular hypertrophy (Fulton index) was observed. RV hypertrophy instigated by increased vascular resistance is primarily a compensatory mechanism but this often leads to RV dysfunction. In accordance with the increased pressure, the lung tissue of high-pressure rats showed perivascular fibrosis and vasoconstriction in pulmonary arteries. Ki-67, a sensitive protein marker strongly associated with cell proliferation was found to be highly expressed in the pulmonary vasculature. This proliferation in the vascular wall and vasoconstriction explains the reason for elevated RV pressure in early PAH. Fascinatingly, the NP peptide treatment improved RV function and pulmonary vascular remodeling in the two-week sugen/hypoxic model. Similarly, in a previous report, inhibition of Akt inhibited muscularization of pulmonary arterioles and reduced right ventricular hypertrophy. These findings suggest the idea that inhibition of Akt nitration with NP could regulate its activation and can prevent downstream cellular proliferative and structural changes in RV and lung vasculature. Moreover, unlike the small molecule inhibitors of Akt, NP peptide can diminish pathological events of nitration mediated Akt activation without affecting normal Akt function. Thus, this is considered to be an important benefit of the targeted antioxidant approach.

Previous reports identified that glycolytic shift (Warburg effect) is a major pathogenic cause, in extreme cases of PAH. FIGS. 4F and 4G showed increased glycolytic and decreased oxidative phosphorylation rate in HPASMCs with SIN-1 treatment. This explains Akt nitration induced metabolic reprogramming from oxidative phosphorylation to glycolysis. NP treatment inhibited Akt nitration and reversed the metabolic reprogramming. Akt activation by peroxynitrite can enhance cellular glucose uptake through the glucose transporter, Glut4, and can increase glycolysis in PAH. In the present invention, Glut4 expression was profiled in different cellular fractions and found it was increased in the membrane/cytosolic fraction. In early two-weeks of PAH, nitrated Akt could perhaps aggravate glucose uptake through increased membrane Glut4 translocation. High level of intracellular glucose influx could upregulate the glycolytic metabolism to meet the demands of increased cellular proliferation. This signifies the Seahorse analysis that PAH shifts glycolysis and mitochondrial respiration in sugen model. Proliferating cells tend to have increased expression of glucose transporters and glycolytic enzymes to compensate for the increased metabolic needs. Inhibition of Akt nitration with NP peptide maintained glucose influx by downregulating the translocation of Glut 4 to the membrane fraction.

In the examples of the present invention, glycolytic enzymes HK-1 and GAPDH showed increased expression in lung tissue of two-weeks sugen/hypoxia. HK1 is the first rate-limiting enzyme of glycolysis, and its overexpression can initiate downstream pathways, amplifying reactions that offer increased ATP to proliferating cells. Also, Akt activation can exacerbate glycolysis by GAPDH phosphorylation and activation in hypoxic stress conditions. Without wishing to limit the present invention to any particular theory or mechanism, it is believed that Akt activation by nitration plays a cardinal role in cellular glucose uptake and increased glycolysis in proliferating cells of early PAH. LDHA converts pyruvate to more lactate in rapid flux metabolism through glycolysis. Increased LDH expression and activity is characteristic of fast-growing cells, and its suppression stimulates mitochondrial respiration and decreases proliferation. An example described herein, shows that nitration of Akt intersects with the aerobic glycolysis pathway in two-weeks of hypoxic sugen model with increased LDHA expression. Increased lactic acid production may result in lactic acidosis, a serious physiological event in PAH. Pyruvate generated in the latter phases of glycolysis is converted to either lactate, via aerobic glycolytic metabolism, or acetyl-CoA, through the oxidation of glucose by PDH. PDH is often considered as the “mitochondrial gate-keeping enzyme”, promoting pyruvate entry into the OXPHOS. Interestingly, inhibition of Akt activation with NP increases mitochondrial enzyme PDH, signifying upregulation of oxidative phosphorylation, and a reduction of lactate accumulation. Thus, in early PAH model, the NP peptide treatment efficiently reversed the metabolic reprogramming from glycolysis to oxidative phosphorylation and prevented cellular proliferative changes.

GSK3β is a serine-threonine kinase, regulating a variety of cellular functions like cell division, proliferation, and differentiation. Akt is one of the critical regulators of GSK-3, and phosphorylation and inactivation of GSK-3 represent the anti-apoptotic effects of Akt. In the PAH model, Akt nitration did not upregulate phosphorylation of Ser9 in GSK3β, but the total expression of GSK3β was increased. GSK3β phosphorylation is controlled by phosphorylated Akt, but in early PAH Akt phosphorylation was found not activated, this could perhaps be the reason for no change in phospho GSK3β expression. Elevation of GSK3β inversely correlates with glycogen synthase. Inhibition of glycogen synthase downregulates glycogen synthesis and could produce more LDH, and oxaloacetate for aerobic respiration and anaplerosis. But the NP peptide by preventing Akt activation decreased GSK3β expression and maintained glycogen synthesis. On the other hand, G6PD, the rate-limiting enzyme of PPP, was found to be decreased. This could downregulate PPP and redirect the metabolism towards increased glycolysis similar to that found in the recent Complex III deficient model. In accordance with this, a report in PAH patients suggests that G6PD deficiency potentiates hemolysis and disease progression. In the two-week sugen/hypoxic model, nitro Akt activation maintained G6PD expression and corrected the PPP, thus balancing glycolytic metabolism to normal.

Downregulation of glycogen synthesis and PPP in early PAH lungs shifted the metabolism towards glycolysis and perhaps produced increased pyruvate. As provided herein, pyruvate could have activated the PC and produces increased oxaloacetate for the TCA cycle. This could result in increased anaplerosis. Anaplerosis by PC and glutaminolysis are the reason for pulmonary vascular constriction and increased proliferation in PAH. Increased glycolysis and anaplerosis provides supplementary biomass for cellular proliferation in early PAH. Increased anaplerosis could lead to a reduction in oxidative phosphorylation and causes mitochondrial dysfunction. Two-weeks of sugen/hypoxia increased PGC-1α expression in lung tissue, representing impairment in mitochondrial function and mitochondria biogenesis demand. PGC-1α might be increased for compensating the metabolic reprogramming and mitochondrial dysfunction in early hypoxic stress. Activation of Akt is associated with the regulation of PGC-1α. In the early PAH model, activation of Akt by Tyr-350 nitration may have upregulated PGC-1α expression. Excitingly, the prevention of Akt nitration with NP downregulated PGC-1α expression representing improved mitochondrial functioning and a reduction in cellular anaplerosis.

In summary, the results described herein suggest that inhibition of Akt nitration with targeted NP peptide can control metabolic reprogramming and pathophysiological events in early PAH. Specifically, NP prevented the activation of Akt by nitration, which controlled a series of cascades, including the balancing of intracellular glucose influx, lactic acidosis, and anaplerosis. Improved glycogenesis, PPP, and oxidative phosphorylation maintained cellular metabolic balance and decreased proliferation. Suppressing cellular proliferation reduced RV hypertrophy and pulmonary vascular remodeling. These dramatic events controlled vascular flow and regulated RV pressure back to normal.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the FIG.s presented in this patent application are drawn to scale, including the angles, ratios of dimensions, etc. In some embodiments, the FIG.s are representative only and the claims are not limited by the dimensions of the FIG.s. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A conjugated peptide with affinity for or targeted to Akt comprising a peptide conjugated to an antioxidant moiety, wherein the conjugated peptide prevents or inhibits protein oxidation of the targeted Akt protein.

2. The conjugated peptide of claim 1, wherein the peptide comprises SEQ ID NO: 1.

3. The conjugate peptide of claim 1, wherein the peptide has an affinity for proximity to Tyr 350 residue of Akt.

4. The conjugated peptide of claim 1, wherein the antioxidant moiety is nitroxide (3-Carboxy-2,2,5,5-tetramethyl-3-pyrroline-1-yloxy) that is covalently attached to a free N-terminal amine of the peptide.

5. The conjugated peptide of claim 1, wherein the conjugated peptide inhibits nitration of Akt.

6. The conjugated peptide of claim 5, wherein the conjugated peptide inhibits Akt nitration which maintains glucose influx and provides a useful treatment option for controlling vascular proliferation in pulmonary arterial hypertension (PAH).

7. (canceled)

8. (canceled)

9. The conjugated peptide of claim 1, wherein the conjugated peptide is for the treatment of pulmonary arterial hypertension (PAH).

10. (canceled)

11. A composition comprising a conjugated peptide with affinity for or targeted to Akt, wherein the conjugated peptide comprises a targeted peptide conjugated to an antioxidant moiety.

12. The composition of claim 11, wherein the peptide comprises SEQ ID NO: 1 having affinity for proximity to Tyr 350 residue of Akt.

13. The composition of claim 11, wherein the antioxidant moiety is nitroxide (3-Carboxy-2,2,5,5-tetramethyl-3-pyrroline-1-yloxy) that is covalently attached to a free N-terminal amine of the peptide.

14. The composition of claim 11 wherein the conjugated peptide inhibits nitration of Akt.

15. The composition of claim 14, wherein the conjugated peptide inhibits Akt nitration which maintains glucose influx.

16. The composition of claim 14, wherein the conjugated peptide inhibits Akt nitration which provides a useful treatment option for controlling vascular proliferation in pulmonary arterial hypertension (PAH).

17. (canceled)

18. The composition of claim 11, wherein the conjugated peptide is for the treatment of pulmonary arterial hypertension (PAH).

19. (canceled)

20. A method of treating pulmonary arterial hypertension (PAH) in a patient in need thereof, the method comprising administering an effective amount of a conjugated peptide to the patient to inhibit or prevent Akt nitration, wherein the conjugated peptide comprises two parts:
a) a peptide portion comprising SEQ ID NO: 1 with affinity for proximity to Tyr 350 residue of Akt; and
b) an antioxidant conjugated to the peptide;
wherein, the conjugated peptide inhibits nitration of Akt.

21. The method of claim 20, wherein the antioxidant is nitroxide (3-Carboxy-2,2,5,5-tetramethyl-3-pyrroline-1-yloxy), and is covalently attached to a free N-terminal amine of the peptide

22. The method of claim 20, wherein the conjugated peptide inhibits Akt nitration which maintains glucose influx.

23. The method of claim 20, wherein the conjugated peptide inhibits nitration early in development of vascular proliferation of PAH.

24. The method of claim 20, wherein treatment with the conjugated peptide reverses the metabolic reprogramming from glycolysis to oxidative phosphorylation and prevents cellular proliferative changes.

25. The method of claim 20, wherein the conjugated peptide protects proteins from oxidation.


SEQUENCE LISTING (4 sequences)

SEQ ID NO: 1 - protein, 5 aa, Homo sapiens
SRLRS

SEQ ID NO: 2 - protein, 13 aa, Homo sapiens
  MISC_FEATURE (7)..(7): Y nitration
CGRLPFYNQDHEK

SEQ ID NO: 3 - protein, 13 aa, Homo sapiens
CGRLPFYNQDHEK

SEQ ID NO: 4 - protein, 5 aa, Homo sapiens
  misc_feature (1)..(1): Xaa can be any naturally occurring amino acid
  misc_feature (3)..(3): Xaa can be any naturally occurring amino acid
  misc_feature (5)..(5): Xaa can be any naturally occurring amino acid
XRXRX